(19) United States
(12) Patent Application Publication
(10) Pub. No.: US 2008/0086071 A1
(43) Pub. Date: Apr. 10, 2008

(54) Therapeutic Device for Treating Soft Tissue Swelling and Fibrosis

(76) Inventor: Kathy Jo Weatherly, Brookfield, WI (US)

Correspondence Address: WHYTE HIRSCHBOECK DUDEK S C, 555 EAST WELLS STREET, SUITE 1900, MILWAUKEE, WI, 53202, US

(21) Appl. No.: 11/952,642

(22) Filed: Dec. 7, 2007

Related U.S. Application Data
(62) Division of application No. 11/068,517, filed on Feb. 28, 2005.
(60) Provisional application No. 60/548,317, filed on Feb. 27, 2004.

(51) Int. Cl. A61L 15/00

(52) U.S. Cl. 602/75

(57) ABSTRACT

An apparatus for treating swelling and tissue induration comprises a therapeutic garment comprising quilting seams which form pockets or channels, wherein the pockets provide gradients that are approximately parallel channels or have a width that tapers from a wide to a narrow configuration and wherein a flexible elastic or elastomeric foam filler at least partially fills the pockets or channels and an outer covering attached to or overlaying the therapeutic garment, wherein the outer covering is adapted to provide constrictive force to the therapeutic garment.

### CROSS REFERENCE TO RELATED APPLICATIONS

This application is a divisional of pending U.S. Pat. application Ser. No. 11/068,517 filed Feb. 28, 2005, which claims priority to expired U.S. Provisional Patent Application No. 60/548,317, filed Feb. 27, 2004, both of which are hereby incorporated by reference in their entirety.

### BACKGROUND OF THE INVENTION

This invention relates generally to resolving swelling and induration of the soft tissue, and more specifically to methods and apparatus for enhancing and directing interstitial fluid movement, stimulation of the lymphatic system and reduction of fibrin formation.

Soft tissue swelling/edema and fibrosis are classic manifestations of the inflammatory process associated with acute/sub-acute injury, repetitive stress to tendons and/or ligaments, and degenerative conditions affecting the joints. An accumulation of plasma proteins and cellular waste within the interstitial fluid resulting from an impairment of the lymphatic system, presents as indurate/fibrotic tissue and swelling, most commonly affecting a body segment or quadrant.

According to Steven Stovitz, MD, CAQ, Sports Medicine “Strain injuries are the result of excessive tension on the muscle, that results in a tear, followed by inflammation, clearing of debris and finally regeneration of the muscle.”(5) Inflammation is the tissue response to injury . . . the major symptoms of inflammation include localized redness, swelling, heat, and pain. Swelling is the result of a net change of the tissue balance from a negative to positive state. Edema occurs when the rate of fluid filtration out of the capillary bed exceeds the ability of the lymphatic drainage system to return the filtered fluid to the vascular system. Swelling poses a threat because it compromises diffusion and osmosis in microcirculation. Added fluid in the interstitial space increases the distances that substances such as, nutrients and oxygen from the arterioles to the cells and bacteria, proteins and dead cells to the lymphatics must travel, this disruption in fluid dynamics creates a state of cellular starvation. As interstitial pressure rises, the gradient against which the lymphatics must pump is diminished, and the volume of fluid they pump increases, fluid begins to accumulate in the tissue and expand this space. Because an increase in interstitial fluid pressure opposes fluid filtration, the driving forces that promote filtration will eventually be balanced and further accumulation of fluid in the interstitial compartment will cease when lymph flow equals filtration. The lymphatic system role is to remove cellular waste, including bacteria, dead cells and excess fluid. If the tissue damage is severe enough, some of the local structural proteins may be solubilized as well. Normally the fluid in the lymphatic vessels is scanty and contains very little protein. This concentration of protein changes when the initial lymphatics drain an area where soluble proteins have accumulated because of inflammation.

Inflammation leads to fibrin formation due to fibrosclerotic tissue, which impairs endothelial cells in lymphatic capillaries, thus inhibiting the intrinsic contractions of the lymphatic collectors. According to Weissleder, “the protein-rich interstitial fluid is replaced by fibrotic tissue . . . and . . . the possible long-term effects of cell proliferation caused by a chronic, inflammatory process”. (Weissleder and Schuchhardt, Lymphedema Diagnosis and Therapy, pg 40-42, 3rd Ed.—Kolhn: Viavital-Verl, 2001). Casely-Smith elaborated on the “damaging phenomenon: Excess fibrosis caused by high-protein edema impedes the flow of fluid and proteins through the tissue channels to the initial lymphatic. From the point of view of healing and re-growth of the lymphatic system, it is important to note that even 1 mm of fibrotic tissue will prevent the passage of regenerating lymphatics, “. . . excess fibrosis will also hamper the entrance of macrophages to the region . . . ” according to Casely-Smith (Modern Treatment for Lymphedema, pg. 66, Henry Thomas Laboratory, University of Adelaide, 1994). This disruption encourages plasma proteins to become clustered and bonded into an abnormal tissue that the initial lymphatic system cannot reabsorb.

Lymphatic vessels, unlike blood vessels, only carry fluid away from the tissues. The smallest of these vessels are the lymph capillaries, which begin in the tissue spaces as blind-ended sacs. Lymphatic capillaries are found in all regions of the body except the bone marrow, central nervous system, and tissues, such as the epidermis, that lack blood vessels. The wall of the lymphatic capillary is composed of endothelium in which the simple squamous cells overlap to form a simple one-way valve. The gaps between the flap valves readily permit the entry of interstitial fluid but close to prevent reflux. Lymphatic capillaries are bonded to the tissue by filaments, as tissue stretches, gap junctions are elongated so that the lumen of a capillary remains open, creating a negative internal pressure, thus drawing in interstitial fluid. Under physiologic conditions of flow and pressure, lymph flows from the superficial to the deeper network. The inner network, in turn, drains lymph into somewhat larger vessels where they merge into larger collecting lymphatic vessels. Intrinsic contractions promote the one-way flow of fluid (lymph) toward the large ducts.

The lymphatic system is solely responsible for balancing the concentration of plasma proteins in the interstitial space since the venous system can not absorb larger molecules. Plasma proteins, which are hydrophilic, attract and sequester water molecules thereby inhibiting osmotic absorption of water by the blood capillaries; the net result is edema (swelling) of the tissue. Reduction of plasma protein clotting, stagnation and concentration will directly effect the proportion of fluid retention in a given body area and the health of the tissue. In addition, high protein concentration in the interstitial is perceived as foreign bodies, causing chronic inflammation and subsequent proliferation of connective tissue resulting in the development of fibrosclerosis.

In one preferred embodiment, the invention is an apparatus for alleviating swelling in a patient. The apparatus comprising a therapeutic garment comprising quilting seams which form pockets or channels, wherein the pockets or channels have a width that may either parallel or taper from a wide to narrow configuration. A flexible elastic or elastomeric foam filler at least partially fills the pockets or channels. An outer covering, attached to or overlaying the therapeutic garment, is adapted to provide stabilizing and constrictive force to the therapeutic garment.

### BRIEF SUMMARY OF THE INVENTION

In one preferred embodiment, the invention is an apparatus for alleviating swelling in a patient, the apparatus comprising a therapeutic garment comprising quilting seams which form pockets or channels, wherein the pockets or channels have a width that may either parallel or taper from a wide to narrow configuration. A flexible elastic or elastomeric foam filler at least partially fills the pockets or channels. An outer covering, attached to or overlaying the therapeutic garment, is adapted to provide stabilizing and constrictive force to the therapeutic garment.

In another preferred embodiment, the invention is a method for alleviating swelling in a patient, the method places a therapeutic garment over a swollen part of a patient's body, wherein the therapeutic garment comprises quilting seams which form pockets or channels, wherein the pockets or channels have a width that either parallels or tapers from a wide to narrow configuration and wherein a flexible elastic or elastomeric foam filler at least partially fills the pockets or channels. An outer covering, which is attached to or overlays the therapeutic garment, is closed such that a stabilizing and constrictive force is transmitted to the therapeutic garment from the outer covering.

### BRIEF DESCRIPTION OF THE DRAWINGS

FIG. 1 is a schematic view of a therapeutic garment in an open position.

FIG. 2 is a schematic view of the therapeutic garment of FIG. 1 in a closed position.

FIG. 3 is a schematic view of an alternative design for the outer covering of the garment.

FIG. 4 is a schematic view of various therapeutic garments designed for use on different parts of a patient's body.

### DETAILED DESCRIPTION OF THE INVENTION

The apparatus of the invention comprises garments providing gradient therapeutic compression. Gradient therapeutic compressions achieved through high to low-pressure ratios created by variation in density, type, size, proportion, and insertion pressure of foams into predetermined pockets or channels created in the fabric. Such gradation is manufactured in a purposeful configuration to facilitate interstitial fluid movement within the tissue based on the core principles of physics as it refers to fluid dynamics. Depending on the body area, gradient therapeutic compression may be created from distal to proximal and/or medial to lateral. Generally, the pockets are made either parallel or to have a narrower width in the high pressure zone and a wider width in the lower pressure areas.

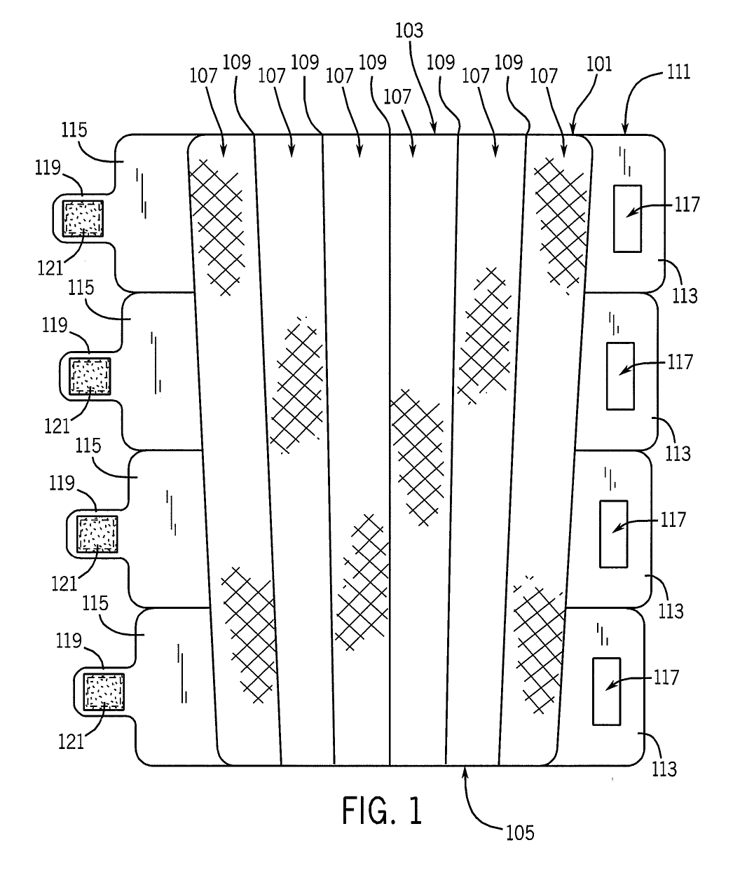

FIG. 1 shows one preferred embodiment wherein the inventive therapeutic garment is a sleeve for use on a patient's limb. The garment 101 has a wide proximal end 103 and a narrow distal end 105. The garment has a number of longitudinal pockets 107. The pockets 107 are formed by quilting the garment 101 with multiple seams 109. Quilting refers to sewing a non-edge seam in a garment to draw a front piece of fabric and a back piece of fabric together. One or more intermediate layers may be interposed between the front and back fabric pieces. The paths of seams 109 are configured to give the desired shapes to the various pockets 107. In this embodiment, the pockets are configured to have a slight taper such that the width of the pocket 107 is wider at the proximal end 103 than at the distal end 105. This configuration creates a gradient pattern that provides for a pressure differential between the high pressure distal end 105 and the low pressure proximal end 103.

In a preferred embodiment, the seams of the garment form roughly parallel pockets. In another preferred embodiment, the seams of the garment form pockets that roughly follow the contours of the shape of the garment. Such contoured pockets typically taper from a relatively wide end to a more narrow end in accordance with the general configuration and anatomy of a body.

The garment 101 comprises a suitable fabric, preferably a fabric with elasticity to hold its shape and provide some therapeutic compression to the body part being treated. The fabric should also be washable, durable and should breathe for comfort. Polyester/lycra fabrics with antimicrobial, wicking, and UV protection properties have been found to be satisfactory for this purpose.

The pockets 107 are at least partially filled with a suitable soft resilient filler. Typically, the filler will be a flexible foam made from an elastic or elastomeric polymer. Such flexible foams are well known in the art and include, for example, polyurethanes, polyethylenes, polypropylenes, vinyl polymers and the like. Satisfactory foams include polypropylene foam of varying sizes and densities including (High Resilient) HR33, HR70, HR 90, Visco-elastic, and Confor ergonomic foams. Further augmenting the gradient pattern, additional pressure differentials are created by the size, density and rebound of the foam products to additionally apply a multiplicity of foam edges into the soft tissue to disrupt abnormal accumulation and bonding of plasma protein. Additionally, foam indention into the tissue create a slow stretch to the skin; physiologically opening the initial lymphatics to facilitate uptake of plasma proteins, cellular waste, bacteria and excess fluid accumulation.

The filler can be inserted into the pockets in any convenient manner, such as by manual stuffing, pouring or by air pressure. The quilting seams may also hold one or more layers of foam filler in place between the front and the back pieces of fabric.

Preferably, the inventive garment also comprises an outer covering 111. Covering 111 can be affixed to garment 101, such as by sewing, or may be a separate item adapted to overlay garment 101. Typically, a hooked fastener-receptive stretch polypropylene outer covering is donned over the device to allow for individualized fit and alteration of the device size as edema resolves. The outer covering is adapted to provide constrictive force to the garment. Said outer covering is designed in a manner to apply equalized torque over the length of the device to provide joint stabilization and support of soft tissues.

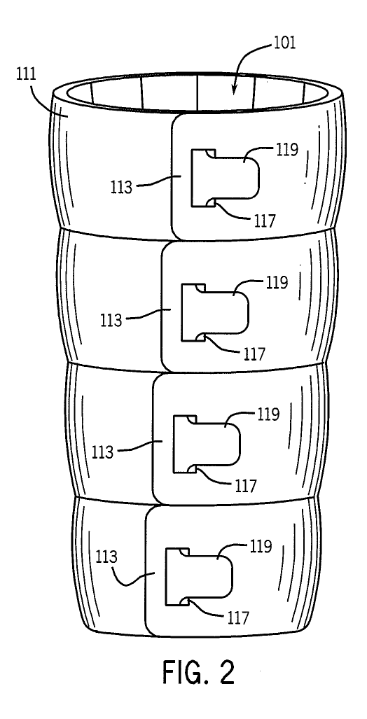

Covering 111 typically has flaps with fasteners. The preferred fastener is a hooked fastener-type fastener because of the ease of adjustment, but other types of fasteners, such as buckles, snaps, belts, straps and the like may be used. The term “hooked fastener” is used to connote various types of fasteners having plastic hooks, such as hook and loop fasteners, hook and pile fasteners, hook and clasp fasteners, and hook bearing surfaces designed to adhere to other fabrics. Such hooked fasteners are typically made from nylon. Hooked fasteners are generally available under the trademark Velcro®. As shown in FIG. 1, covering 111 has a number of female flaps 113 having slots 117. Covering 111 also has a number of male flaps 115 having tabs 119. Tabs 119 bear hooked fastener patches. Covering 111 is closed over garment 101 as shown in FIG. 2. Tabs 119 are inserted through the slots 117 of female flaps 113. The hooked fastener patches 121 (not shown in FIG. 2) catch on a mating section of hooked fastener or, more preferably, onto a hooked fastener accepting fabric on the outside surface of covering 111. One example of a suitable hooked fastener accepting fabric is FabriFoam®.

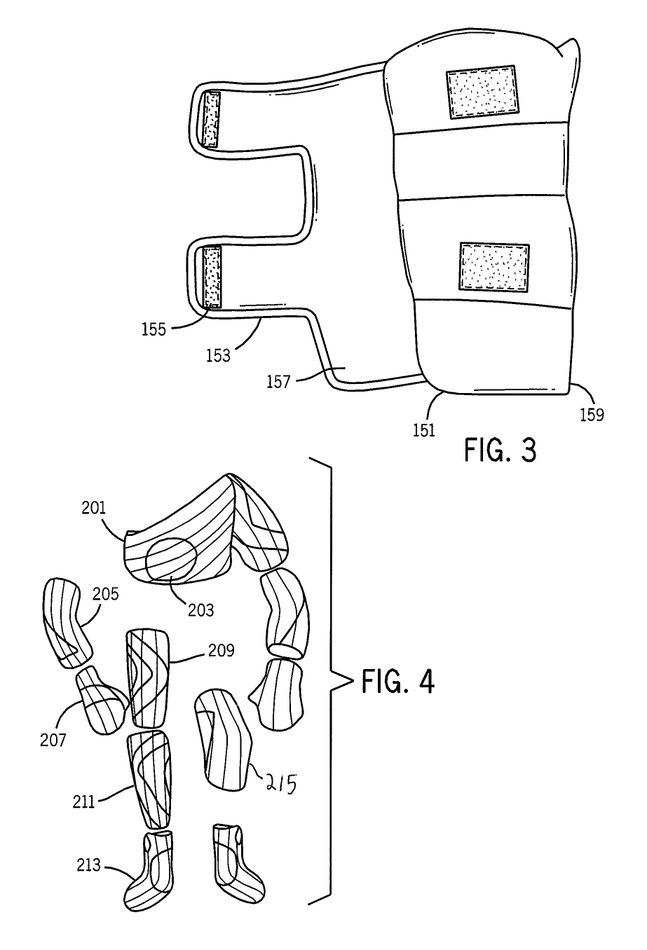

FIG. 3 shows another preferred embodiment of the outer covering. The covering 151 has multiple I-flaps 153. The I-flaps 153 are arranged such that they interlace with each other when covering 151 is closed. Flaps 153 have a hooked fastener patch 155 which will engage with a mating hooked fastener patch, or a hooked fastener accepting fabric 159 on the outside of covering 151 when the covering is closed. Covering 151 preferably has a soft padding 157 on the inside surface to improve patient comfort.

In one preferred embodiment, the outside covering would have a second closure means, such as a zipper. The second closure method would allow a patient to remove the covering without disturbing the therapeutic compression of the covering as set by a therapist.

FIG. 4 shows typical embodiments of the inventive garment for various body parts. Garment 203 is designed to apply compression to the chest and upper shoulder. Note that the seams 201 are horizontal and provide a medial/lateral pressure gradient. Garment 205 is adapted for use on an elbow. Garment 207 is designed for use on a hand or wrist. Garment 209 is designed for wearing on the thigh. Garment 211 is designed for wearing around the calf. Garment 213 is designed for use on the foot or ankle. Garment 215 is designed for use on the knee.

The inventive garments provide passive gradient compression, pressure differentials, lymphatic uptake and fibrosis resolution with out the use of external mechanical pumps or active ROM/MM contractions by the wearer to obtain therapeutic edema reduction.

Use of the inventive garment is simple and convenient. A garment having pockets which provide a pressure gradient is placed over an area of the body which has swelling. The outer covering is placed around the pocketed garment and closed to the appropriate tightness. The primary fasteners, typically hooked fastener, hold the outer covering at the proper tension set by the therapist or wearer. The therapeutic garment can be removed by opening the primary fasteners of the outer covering, or, alternatively, by opening a secondary fastener.

What is claimed is:

1. A method for alleviating swelling and tissue induration in a patient, the method comprising:
a) placing a therapeutic garment over a swollen part of a patient's body, wherein the therapeutic garment comprises quilting seams which form pockets or channels, wherein the pockets or channels are either parallel or taper from a wide to a narrow configuration;
b) closing an outer covering attached to or overlaying the therapeutic garment such that a constrictive force is transmitted to the therapeutic garment from the outer covering.

2. The method of claim 1 wherein the pockets or channels taper and the taper substantially conforms to the contours of a body part that the apparatus is adapted to compress.

3. The method of claim 1 wherein the foam filler comprises a multiplicity of foam edges.

4. The method of claim 1 wherein the therapeutic garment is adapted to provide compressive pressure to body tissue underlying the garment.

5. The method of claim 1 wherein a pressure differential gradient is formed by providing different amounts of pressure to different locations of the underlying tissue.

6. The method of claim 1 wherein the apparatus is adapted for use on a body limb and having a proximate end and a distal end.

7. The method of claim 1 wherein higher pressure is applied to the limb at the distal end of the apparatus than at the proximate end of the apparatus.

8. The method of claim 1 wherein the outer covering further comprises at least one fastener.

9. The method of claim 1 wherein the fastener is a hook fastener.
